(19) United States
(12) Patent Application Publication
(10) Pub. No.: US 2026/0092255 A1
(43) Pub. Date: Apr. 2, 2026

(54) Cell Based Assays for Botulinum

(71) Applicant: BioMadison, Inc., Del Mar, CA (US)

(72) Inventors: Ward Tucker, Monona, WI (US); Francis Mark Dunning, Madison, WI (US); Tim Piazza, Del Mar, CA (US)

(21) Appl. No.: 18/920,316

(22) PCT Filed: Apr. 20, 2023
(86) PCT No.: PCT/US2023/019300

§ 371 Date: Oct. 18, 2024

Related U.S. Application Data
(60) Provisional application No. 63/333,282, filed on Apr. 21, 2022.

(51) Int. Cl. C12N 5/0793; G01N 33/50; G01N 33/58

(52) U.S. Cl. CPC C12N 5/0619; G01N 33/5091; G01N 33/582; C12N 2510/00; G01N 2333/33

(57) ABSTRACT

Serotype-specific cell based assays for the characterization of Clostridia botulinum neurotoxins and genetically modified cells utilized in such assays are described. Such assays and genetically modified cells can discriminate between different serotypes of clostridium neurotoxins having the same SNARE protein substrate specificity, despite a lack of such discrimination in the parental cell line. This discrimination is achieved without the use of serotype-specific neutralizing antibodies and without the use of competing heavy chains derived from a Clostridia botulinum neurotoxin.

This application claims the benefit of PCT Application No. PCT/US2023/019300, filed on Apr. 20, 2023, which claims the benefit of claims the benefit of U.S. Provisional Patent Application No. 63/333,282, filed on Apr. 21, 2022. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is cell based assays for proteases, in particular botulinum neurotoxins.

### BACKGROUND

The following description includes information that may be useful in understanding the present invention. It is not an admission that any of the information provided herein is prior art or relevant to the presently claimed invention, or that any publication specifically or implicitly referenced is prior art.

The background description includes information that may be useful in understanding the present invention. It is not an admission that any of the information provided herein is prior art or relevant to the presently claimed invention, or that any publication specifically or implicitly referenced is prior art.

Botulinum neurotoxins (BoNTs) are produced by Clostridium botulinum, and are among the most potent toxins known. These toxins are a well-recognized source of food poisoning, often resulting in serious harm or even death of the victims. There are seven structurally related botulinum neurotoxins or serotypes (BoNT/A-G), each of which is composed of a heavy chain (≈100 KD) and a light chain (≈50 KD). The heavy chain mediates toxin entry into a target cell through receptor-mediated endocytosis. Once internalized, the light chain is translocated from the endosomal vesicle lumen into the cytosol, and acts as a zinc-dependent protease to cleave proteins that mediate vesicle-target membrane fusion (“substrate proteins”).

These BONT substrate proteins include plasma membrane protein syntaxin, peripheral membrane protein SNAP-25, and a vesicle membrane protein synaptobrevin (Syb). These proteins are collectively referred to as the SNARE (soluble N-ethylmaleimide-sensitive factor attachment protein receptor) proteins. Cleavage of SNARE proteins blocks vesicle fusion with plasma membrane and abolishes neurotransmitter release at neuromuscular junction. Among the SNARE proteins, syntaxin and SNAP-25 usually reside on the target membrane and are thus referred to as t-SNAREs, while synaptobrevin is found exclusively with synaptic vesicles within the synapse and is called v-SNARE. Together, these three proteins form a complex that is thought to be the minimal machinery to mediate the fusion between vesicle membrane and plasma membrane. BoNT/A, E, and C1 cleave SNAP-25, BoNT/B, D, F, G cleave synaptobrevin (Syb), at single but different sites. BoNT/C also cleaves syntaxin in addition to SNAP-25.

Due to their threat as a source of food poisoning, and as bioterrorism weapons, there is a need to accurately and sensitively detect BoNTs. Currently, the “gold standard” method to detect toxins is to perform toxicity assay in mice. This method requires large numbers of mice, is time-consuming, however use of such assays raises ethical concerns. A number of amplified immunoassay systems based on using antibodies against toxins have also been developed. However, since activity of a BoNT depends on both proteolytic activity and appropriate transfer across the cell membrane and subsequent processing, conventional immunoassays directed to the neurotoxin itself are not adequate.

HPLC separation and immunoassays can be used to detect specific peptide fragments generated by proteolytic activity of these toxins, however such methods are time-consuming and complex. Therefore, there is a need for new and improved methods and compositions for detecting BONTs.

Cell based assays based on Forster Resonance Energy Transfer (FRET) measurements of cells carrying reporting peptides incorporated FRET pairs of fluorophores separated by peptides corresponding to the scissile portion of SNARE proteins have been used to detect BoNTs. In such FRET assays, two fluorogenic amino acid derivatives or fluorescent peptides are separated by a short synthetic peptide (typically 12-35 amino acids) that contain the scissile portion of a SNARE protein substrate of a BoNT. In the intact reporting peptide fluorescence signal of one of these fluorescent moieties (i.e., the donor fluorophore) is quenched by the other fluorescent moiety (the acceptor). At the same time an increase in fluorescence is observed from the acceptor fluorophore on excitation of the donor fluorophore. Cleavage of the peptide separates the fluorophores, such that a decrease in FRET (i.e., an increase in fluorescence from the donor fluorophore and a decrease in fluorescence from the acceptor fluorophore) can be detected. Fluorescence quenching assays utilize a very similar approach, with FRET energy transfer to the acceptor moiety not resulting in fluorescence. In such quenching assays, the loss of FRET observed on separation of the donor and acceptor moieties can be characterized by an increase in fluorescence observed from the donor fluorophore.

FRET assays have been successfully used for detecting BoNTs. (See e.g., US Pat. App. No. 2004/0191887 to Chapman, filed Oct. 28, 2003, US Pat. App. No. 2006/0134722 to Chapman, filed Dec. 20, 2004, U.S. Pat. No. 7,208,285 to Steward (April 2007), U.S. Pat. No. 7,183,066 to Fernandez-Salas (February 2007), and application US2011/0033866 (publ. February 2010), and have been adapted for use in living cells. All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Although some success has been demonstrated in applying FRET assays to detection of BoNTs, the sensitivity and specificity are still undesirable for many purposes. Alternatively, cell-based assays that utilize fluorophore pairs that are arranged so that no significant FRET occurs have also been used. (See e.g., U.S. Pat. No. 9,274,121 to Atapattu and Tucker).

A distinct limitation of such cell based assays is that a native botulinum neurotoxin substrate or a reporting construct incorporating such a protein is readily cleaved by more than one botulinum neurotoxin serotype. For example, A, C, and E serotypes are capable of cleaving SNAP25 and reporting constructs incorporating SNAP25. Similarly, B, D, F, and G serotypes are capable of cleaving synaptobrevin and reporting constructs incorporating synaptobrevin.

Thus, there is still a need for cell based assays directed to BoNTs that can provide distinction between different BONT serotypes.

### SUMMARY OF THE INVENTION

The inventive subject matter provides compositions and methods utilized in characterizing botulinum neurotoxins (BoNTs). Surprisingly, Inventors have found that neuroblastoma cell lines that have been genetically modified to produce a reporting peptide or reporting construct that is sensitive to a botulinum neurotoxin can demonstrate serotype specificity that is not exhibited by the parental cell line.

Embodiments of the inventive concept include a cell produced by genetic modification of a parental neuronal cell line and that include a reporting construct, where the reporting construct incorporates a peptide that is a substrate for both BoNT/A and BoNT/E. Such a cell is more than 100-fold more selective for intoxication by serotype A botulinum neurotoxin (BoNT/A) than by serotype E botulinum neurotoxin (BoNT/E), while the parental cell line is equally susceptible to both BoNT/A and BoNT/E. The parental neuronal cell line is a human neuronal or neuroblastoma cell line, for example LA-N-2, SH-SY5Y, N2a, SiMa, NS-20Y, NIE-115, NG108-C15, NSC-34, NSC-19, M4b, CNh, G4b, HT-22, or PC12. In some embodiments the cell remains viable after exposure to polysorbate 20 or polysorbate 80 at a concentration of 0.017% w/v for up to 72 hours.

Embodiments of the inventive concept include a method of selectively quantifying BoNT/A by obtaining a first emission measurement from a genetically modified cell as described above at a first wavelength, obtaining a second emission measurement from the cell at a second wavelength, and dividing the first measurement by the second measurement to obtain a first emission ratio. The cell is then contacted with a solution containing a botulinum neurotoxin. After a period of time (e.g., 24 hours, 48 hours, 72 hours, 96 hours, or intervals between these values), a third measurement is obtained from the cell at the first wavelength as well as a fourth measurement from the cell at the second wavelength. The third measurement is divided by the fourth measurement to obtain a second emission ratio. A reduction of the second emission ratio relative to the first emission ratio is indicative of the presence of a botulinum neurotoxin to which the cell is susceptible in the solution. Such a method can provide a limit of detection of 50 fM or less for BoNT/A. In some embodiments the first emission ratio is not distinguishable from the second emission ratio when the botulinum neurotoxin is BoNT/E at a concentration of 0.5 nM. In some embodiments the solution further comprises one or more pharmaceutical excipients, such as a surfactant (e.g., polysorbate 20, polysorbate 80), that are not removed prior to contacting with the cell.

Embodiments of the inventive concept can include methods of improving a cell-based assay for a botulinum neurotoxin (BoNT) by transfecting a human neuroblastoma cell capable of intoxication by the BoNT to generate a genetically modified cell expressing a reporting construct. A portion of the reporting construct is cleavable by a protease activity of the BoNT. The genetically modified cell expresses a plurality of SV2 isoforms or both GD1a and GT1b gangliosides. The method further includes contacting the genetically modified cell with the BoNT. In some embodiments the genetically modified cell expresses SV2A. SV2b, and/or SV2c, or two of these isoforms. Similarly, in some embodiments the genetically modified cell expresses a plurality of SV2 isoforms, GD1a, and GT1b. The reporting construct can include a membrane binding portion, a linking portion that includes a cleavage site that is a substrate for the BONT's protease activity, and a reporting portion that includes a labile reporter (such as a first fluorophore). Such a labile reporter can be a fluorescent peptide. The reporting portion is coupled to the membrane binding portion via the linking portion, such that cleavage of the cleavage site results in release of the labile reporter into the cell's cytoplasm, where the labile reporter is subject to proteolysis in the cell's cytoplasm within the time course of the cell-based assay. In some embodiments wherein the reporting construct further comprises a reference reporter (such as a second fluorophore) coupled to the membrane binding portion, where the reference reporter remains coupled to the membrane binding portion of cleavage of the cleavage site and is not subject to degradation upon cleavage of the cleavage site. In some embodiments the improvement is a reduction of the EC₅₀ of a culture medium and temperature optimized cell-based assay performed using the genetically modified cell by 50% relative to the EC₅₀ of a culture medium and temperature optimized cell-based assay performed using a murine cell transformed to express the reporting construct.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1: FIG. 1 provides a graphical depicting typical growth of 5E9 and 8F4 genetically modified cell lines.

FIG. 2: FIG. 2 provides a histogram depicting typical viability of 5E9 and 8F4 genetically modified cell lines.

FIG. 3: FIG. 3 provides a graphical depicting typical growth of 2C2 transfected cell line of the 2C2 genetically modified cell line.

FIG. 4: FIG. 4 provides a histogram depicting typical viability of 6h4 2C2 genetically modified cell line.

FIG. 5: FIG. 5 provides a graphical depiction of typical comparative growth curves for 5E9, 8F4, and 2C2 transfected cell lines.

FIG. 6: FIG. 6 provides a graphical depiction of a typical response of a human neuroblastoma cell line that has not been genetically modified to BoNT/A and BoNT/E, as shown by a prior art immunoassay directed to serotype-specific fragments of SNAP25.

FIG. 7: FIG. 7 provides a graphical depiction of a typical response of genetically modified cell line 2C2 to BoNT/A and BoNT/E, shown as YFP/CFP normalized ratio.

FIG. 8: FIG. 8 provides a graphical depiction of a typical comparison of sensitivity BoCell® and 2C2 transformed cell line response to BoNT/E.

FIG. 9: FIG. 9 provides a graphical depiction of a typical comparison of relative sensitivity of BoCell® and 2C2, 5E9, and BF4 transformed cell lines to BoNT/A.

FIG. 10: FIG. 10 provides a graphical depiction of a typical culture time, culture temperature, and cell density effects on EC₅₀ of a cell-based BoNT/A assay utilizing genetically modified human neuroblastoma cells.

FIG. 11: FIG. 11 provides typical photomicrographs depicting expression of SV2A, SV2B, and SV2C in the 8F4 human neuroblastoma genetically modified cell line.

FIG. 12: FIG. 12 provides typical photomicrographs depicting expression of SV2A, SV2B, and SV2C in the 5E9 human neuroblastoma genetically modified cell lines.

FIG. 13: FIG. 13 provides typical photomicrographs depicting expression of SV2A, SV2B, and SV2C in the 8F4 human neuroblastoma genetically modified cell lines.

FIG. 14: FIG. 14 provides typical photomicrographs depicting expression of SV2A, SV2B, and SV2C in the 9C5 human neuroblastoma genetically modified cell line.

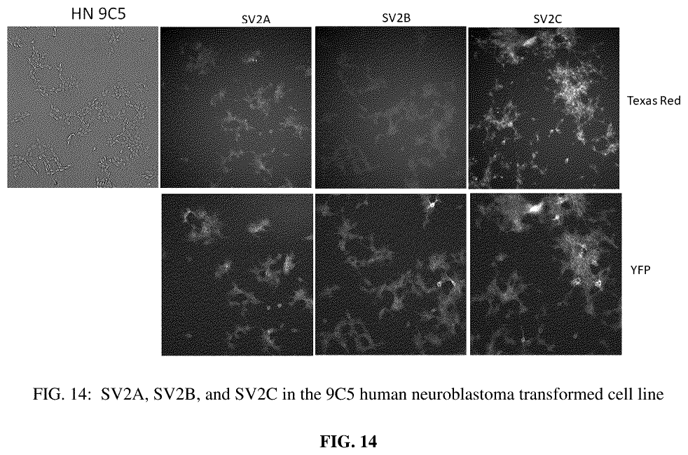

FIG. 15: FIG. 15 provides typical photomicrographs depicting expression of SV2A, SV2B, and SV2C in a BoCell® cell line expressing the same reporting construct as in human neuroblastoma transformed cells of FIGS. 11 to 14.

FIG. 16: FIG. 16 provides typical photomicrographs depicting SV2 isoform expression in 2C2 transformed human neuroblastoma cells using polyclonal antibodies.

FIG. 17: FIG. 17 provides typical photomicrographs depicting ganglioside GD1a and GT1b content of human neuroblastoma cells, human neuroblastoma genetically modified to express a BoNT/A sensitive reporting construct, and BoCell® cells expressing the same reporting construct.

FIG. 18: FIG. 18 shows the results of exposure of BoCell® cells and genetically modified human neuroblastoma cells expressing the same reporting construct to polysorbate 20 (P20, in the upper portion of FIG. 18) and polysorbate 80 (P80, in the lower portion of FIG. 18) at different concentrations. Cell viability was characterized by measuring fluorescence emissions of the fluorescent moieties of the reporting construct. The left side of FIG. 18 shows fluorescence emissions from the portion of reporting construct that is degraded upon exposure of the cells to BoNT/A. The right side of FIG. 18 shows fluorescence emissions from the cells normalized as described above

### DETAILED DESCRIPTION

The following description includes information that may be useful in understanding the present invention. It is not an admission that any of the information provided herein is prior art or relevant to the presently claimed invention, or that any publication specifically or implicitly referenced is prior art.

The present invention provides compositions and methods for performance of selective cell based assays for botulinum neurotoxins (BoNTs). These include and incorporate cells that have been genetically modified to express a reporting construct, where a portion of the reporting construct can act as a substrate for the proteolytic activity of a BoNT that is to be characterized. Surprisingly, Inventors have found that such genetically modified cells can exhibit a previously unobserved high degree of selectivity between different BoNT serotypes that utilize the same SNARE protein as a substrate, and that such selectivity is a result of cellular characteristics and functions rather than a characteristic of the reporting construct.

In some embodiments the genetically modified cells are derived from LA-N-2 cells (i.e., LA-N-2 cells are the parental cell line). In some embodiments the genetically modified cells are derived from SH-SY5Y cells (i.e., SH-SY5Y cells are the parental cell line). In some embodiments the genetically modified cells are derived from N2a cells (i.e., N2a cells are the parental cell line). In some embodiments the genetically modified cells are derived from SiMa cells (i.e., SiMa cells are the parental cell line). In some embodiments the genetically modified cells are derived from NS-20Y cells (i.e., NS-20Y cells are the parental cell line). In some embodiments the genetically modified cells are derived from NIE-115 cells (i.e., NIE-115 cells are the parental cell line). In some embodiments the genetically modified cells are derived from NG108-C15 cells (i.e., NG108-C15 cells are the parental cell line). In some embodiments the genetically modified cells are derived from NSC-34, cells (i.e., NSC-34 cells are the parental cell line). In some embodiments the genetically modified cells are derived from NSC-19, cells (i.e., NSC-19 cells are the parental cell line). In some embodiments the genetically modified cells are derived from M4b cells (i.e., M4b cells are the parental cell line). In some embodiments the genetically modified cells are derived from CNh cells (i.e., CNh cells are the parental cell line). In some embodiments the genetically modified cells are derived from G4b cells (i.e., G4b cells are the parental cell line). In some embodiments the genetically modified cells are derived from CNh cells (i.e., CNh cells are the parental cell line). In some embodiments the genetically modified cells are derived from G HT-22 cells (i.e., HT-22 cells are the parental cell line). In some embodiments the genetically modified cells are derived from G PC12 cells (i.e., PC12 cells are the parental cell line). PC12

Surprisingly, the degree of selectivity can differ between a genetically modified cell line and a parental cell line from which the genetically modified cell line is derived, permitting generation of cell lines capable of providing a high degree of discrimination (e.g., a 100-fold or greater difference in EC50) between BoNT serotypes having the same substrate protein specificity (e.g., serotype A and serotype E).

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments a reporting construct expressed in such cells can include (a) a reporter-containing portion coupled to (b) a linking portion that includes a cleavage site, which in turn can act as a substrate for the proteolytic activity of a BoNT that is being characterized, and (c) a membrane-binding portion that is coupled to the reporter-containing portion via the linking portion. Interaction with the BoNT that is being characterized cleaves the reporter-containing portion from a remainder of the reporting construct and releases it into the cytoplasm of the transformed cell. Once released the reporter-containing portion is subject to rapid degradation by proteolytic activity in the cell's cytoplasm.

In preferred embodiments the reporting construct can include a reference reporter that is retained with the membrane-binding portion and is not subject to proteolysis following exposure of the transformed cell to the BoNT being characterized. In such embodiments the reporter-containing portion includes a fluorescent peptide (e.g., a GFP or GFP mutation derived peptide), and the reporting construct can be arranged such that no useful Förster resonance energy transfer (FRET) (e.g., less than 5% energy transfer) occurs between this fluorescent peptide and a fluorophore (e.g., a second and different fluorescent peptide) of the reference reporter. This advantageously permits use of measurements from the reference reporter to normalize measurement from the reporter portion across different test wells. For example, emission from a “reporting” fluorophore that is degraded by proteolysis upon release by BoNT activity can be divided by emission from a “reference” fluorophore that is not subjected to such degradation to correct for differences between cell number, expression levels, etc. between different wells of a test plate utilized in such a cell based assay.

Such transformed cells can be utilized in methods for characterizing BoNTs, qualitatively and/or quantitatively. An example of such a method can include
(i) providing a composition that includes a cell transformed to express a reporting construct as described above
(ii) obtaining a baseline signal from the reporting-containing portion of the reporting construct
(iii) exposing the cell to the BoNT to be characterized
(iv) obtaining a further signal from the reporting-containing portion of the reporting construct after sufficient time has passed for cleavage of the reporting construct by the BoNT and degradation of released reporting portion in the cell's cytosol.
(v) comparing the first emission measurements of step (ii) with the further measurements of step (iv).

Typically, these assays are performed at a temperature of 35° C. to 39° C. (preferably at 37° C.), with the testing interval ranging from 24 hours to 72 hours. Temperature, time, and culture media conditions can be optimized to suit specific testing needs.

The cleaved reporter-containing portion is destroyed or otherwise degraded by the local environment, and presence and/or quantity of the BoNT to be characterized is then evidenced by a reduction in signal from the reporter. In the context of this application, it is contemplated that degradation of released reporter will typically proceed intracellularly by at least one of two pathways, by the ubiquitin-dependent process that targets proteins to the proteasome, or by the autophagy-lysosomal pathway. In one pathway, the proteasome is the enzyme. In the lysosome pathways, it is contemplated that the enzymes of interest are hydrolases, including especially a family of proteases called the cathepsins.

BoNTs suitable for characterization include BoNT/A, BoNT/B, BONT/C, BONT/D, BONT/E, BONT/F, BoNT/G, and BoNT/H. Linking portions of suitable reporting constructs can include a cleavage site with a sequence selected to correspond to substrate specificity of the BONT to be characterized. Contemplated cleavage site sequences can advantageously comprise a SNARE protein, motif, or mutein. “Muteins” of a protein should be interpreted herein as having at least 30% identity with a corresponding native protein, including for example compositions having at least 35%, 40%, 45%, 50%, 55%, 60%, 65%, 70%, 75%, 80%, 85%, 90%, 95% or 98% identity with the native protein. Variations from identity can comprise any or more of additions, deletions and substitutions. Contemplated muteins include fragments, truncates and fusion proteins.

As noted above, a reporter-containing portion of a reporting construct can include a fluorescent protein or peptide, for example a Green Fluorescent Protein (GFP) mutation such as Yellow Fluorescent Protein (YFP), Citrine, Venus, and YPet proteins. The sequence of such fluorescent proteins mentioned herein can be modified to include specific characteristics (e.g., spectral) or be truncated to a desired size.

As noted above, a portion of the reporting construct that remains associated with the cell membrane following cleavage by the BoNT being characterized can include a second fluorescent protein, such as Cyan Fluorescent Protein (CFP), mCherry, or mStrawberry. The emission spectra of such a second fluorophore can be selected for minimal overlap with that of a fluorophore of the reporter-containing portion in order to simplify data normalization.

Any suitable reporting construct can be utilized in cells and methods of the inventive concept. SEQ ID NO. 1 (provided in the accompanying sequence listing) is an example of a suitable reporting construct that provides no useful FRET when intact. This reporting construct includes portion corresponding to SNAP25, flanking spacer peptides, and a YFP portion that is released by the proteolytic activity of BoNT/a and BoNT/E light chains, and is rapidly degraded upon release. This reporting construct also includes a CFP portion that is not degraded following proteolysis by BoNT/A or BoNT/E, the emission of which can be used to normalize emission measurements made of YFP during the course of a cell based assay.

Such a reporting construct can be introduced into a cell by any suitable means, including introduction of a plasmid or viral genome that encodes the reporting construct. Such a plasmid or viral genome can incorporate a transcription promoter positioned to influence transcription of the portion of the plasmid or viral genome encoding the reporting construct. Such a plasmid or viral genome can incorporate additional coding regions that permit screening of transformed cells, such as regions encoding for antibiotic resistance.

In a preferred cell-based assay for characterizing a specified BONT, a cell containing the reporting construct will exhibit a baseline signal from the reporting portion, and then after exposure exhibits a reduced signal from the reporting portion. In a reporting construct incorporating YFP into the reporting portion and utilizing CFP for normalization separately excited YFP emissions (top, Ex500, Em526) and CFP emissions (middle, Ex434, Em470) are collected. These emissions are then background subtracted and the YFP emission is divided by CFP emission to control for cell density and reporter expression in the individual cells. That emission ratio (YFP/CFP, bottom) is how the assay is reported.

Destruction or other degradation of the reporter-containing portion takes place at a much faster rate post-exposure to the BoNT than pre-exposure. In preferred embodiments, it is contemplated that the destruction or other degradation of the reporter containing portion occurs at least 2× (twice) as fast post-exposure as pre-exposure, but more preferably the rate post-exposure rate is at least 5×, at least 10×, at least 100× relative to the pre-exposure rate.

Cells utilized in methods of the inventive concept are selected to be susceptible to intoxication by the BoNT that is to be characterized. Typically, such cells are of neuronal origin. Suitable cell lines include, but are not limited to, LA-N-2, SH-SY5Y, N2a, SiMa, NS-20Y, NIE-115, NG108-C15, NSC-34, NSC-19, M4b, CNh, G4b, HT-22, and PC12. Surprisingly, Inventors have found unexpected differences in performance characteristics between different transformed modified to express the same reporting construct. For example, differences in regard to sensitivity, BoNT serotype selectivity, and sensitivity to surfactants have been found that cannot be accounted for by characteristics of the parental cell line that has been modified.

In some embodiments of the inventive concept, a cell line that is susceptible to BONT intoxication (for example, a neuroblastoma cell line) is genetically modified to express a reporting construct (e.g., the reporting construct represented by SEQ ID NO. 1) that is susceptible to cleavage by proteolytic activity of the light chain of a BoNT of interest. As noted above, such a reporting construct can include a fluorescent moiety that has utility in a cell-based assay (for example, by degrading following scission of the reporting construct or providing a normalizing signal from the reporting construct). Such a reporting construct can be introduced by transformation of the cells with an expression vector, plasmid, or virus that includes genetic information encoding such a reporting construct prepared by and administered by methods as known in the art. Such transformations can be performed by any suitable method, including electroporation, infection via a viral vector (e.g., a lentivirus), direct injection, etc.

Genetically modified cells derived from the cell line can be identified by any suitable means. In some embodiments such cells can be identified by selection for selection markers incorporated into the expression vector, plasmid, or viral genome encoding the construct (for example, by encoding for resistance to an antibiotic). Alternatively, or in addition, genetically modified cells expressing the reporting construct can be identified and isolated by fluorescence activated cell sorting (FACS) utilizing emission from a fluorescent moiety of the expressed reporting construct. For example, droplets containing cells showing fluorescence associated with the reporting construct can be separated and expanded in culture.

As noted above, the Inventors have surprisingly identified cells that exhibit a high degree of selectivity (e.g., showing a 100-fold, 200-fold, 300-fold, 400-fold, 500-fold, 600-fold, 700-fold, 800-fold, 900-fold, 1,000 fold or greater difference if EC₅₀) for a specific BoNT serotype over a different BoNT serotype having the same SNARE protein specificity, where such selectivity is not found in the corresponding cell line prior to transformation. Such cells can be identified by comparison of the results of exposure to a range of concentrations of different BONT serotypes on cleavage of the reporting construct (e.g., by monitoring loss of fluorescence in the fluorescent moiety so released). In some embodiments this can be performed by culture of the genetically modified cells in a series of wells in a microwell plate in the presence of the BONT, followed by fluorescence measurements of the wells. Alternatively, such cells can be isolated using FACS to segregate cells responsive to a desired BONT serotype, for example by contacting the genetically modified cells with a high concentration of the BoNT and measuring a low ratio (e.g., 0.1, 0.05, 0.05, 0.01 or less) of fluorescence between the fluorescent moiety responsive to the BoNT and the fluorescent moiety that is not responsive to the BoNT. Cells within this population that are relatively non-responsive to the non-desired BONT can be identified from this population, for example by performing contacting with a high concentration of the non-desired BoNT and identifying cells that maintain a high ratio (e.g., greater than 50% of that observed for untreated cells) of fluorescence between the respective fluorophores.

In such studies the relative concentrations of the BoNTs can be initially determined using a conventional mouse lethality assay or an alternative methodology that incorporates both cell internalization and processing steps and has been calibrated to provide results comparable to the mouse lethality assay, so as to provide directly comparable results between the BoNT serotypes.

### EXAMPLES

Cells from a human neuroblastoma (human neuroblastoma) cell line were transformed with an expression vector encoding for a reporting construct (SEQ ID NO. 1) that included:
a reporter-containing portion that include a YFP peptide
a linking portion containing a SNAP-25 derived cleavage site
a membrane binding portion coupled to the reporter-containing portion via the linking portion
A control portion containing a CFP peptide and coupled to the membrane binding portion such that the coupling remains following cleavage of the linking portion.
   The same reporting construct is expressed in murine-derived BoCell® A/E cell lines, data from which is provided for comparative purposes.

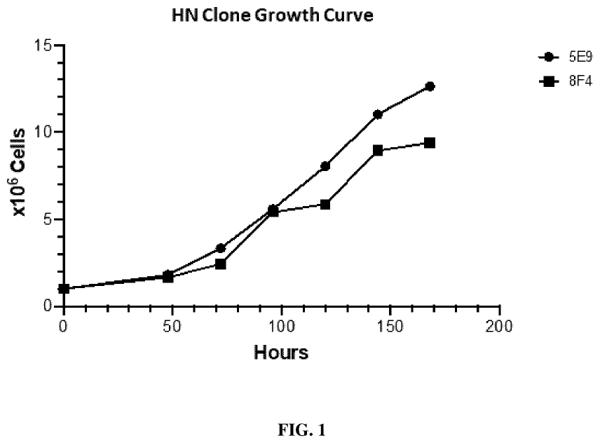

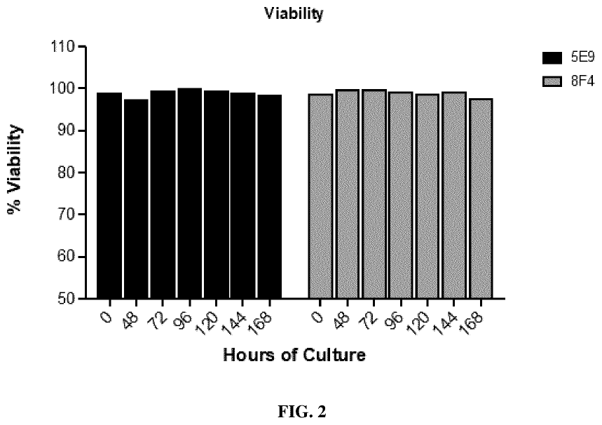

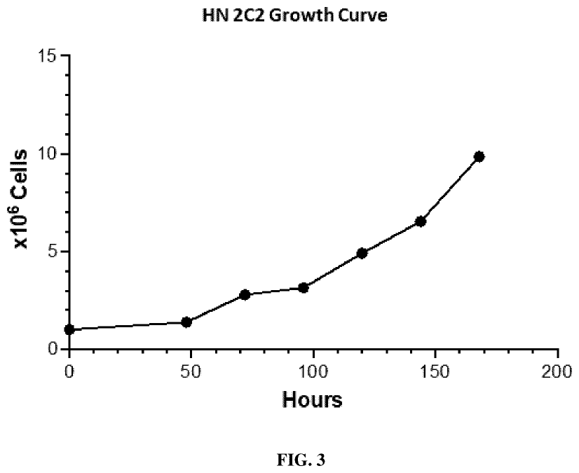

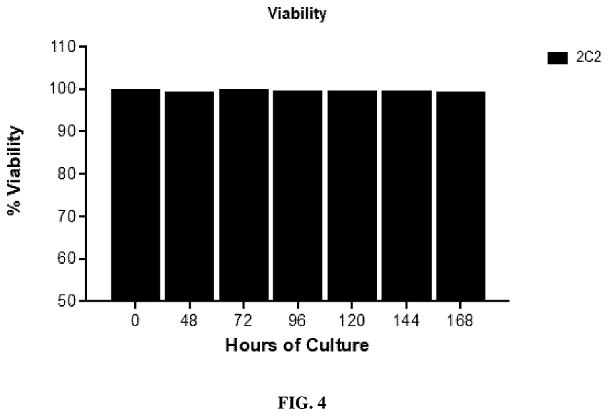

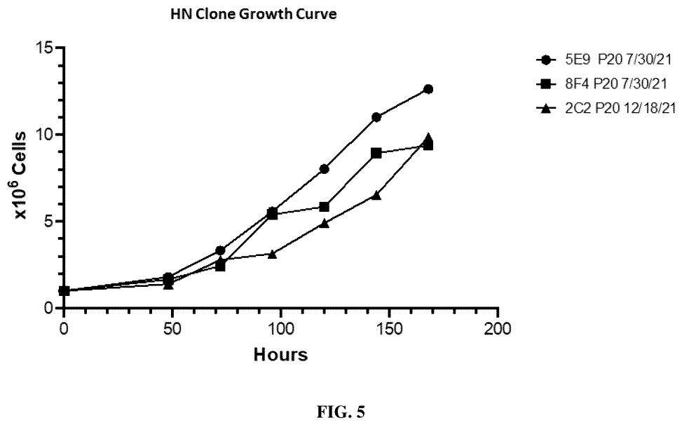

Growth characteristics of several transformed cell lines derived from genetically modified human neuroblastoma (human neuroblastoma) cells and expressing the reporting construct were characterized, as shown in FIGS. 1 to 5. As shown in FIGS. 1 (cell lines 5E9 and 8F4) and 3 (cell line 2C2), genetically modified human neuroblastoma cells expressing a reporting construct as described above show vigorous growth in culture. As shown in FIGS. 2 (cell lines 5E9 and 8F4) and 4 (cell line 2C2, viability of the genetically modified cell lines remains at 95% or more through at least 168 hours of culture. FIG. 5 provides comparative results for genetically modified human neuroblastoma cell lines 5E9, 8F4, and 2C2, showing similar rates of growth over time. These genetically modified cell lines were found to provide EC₅₀s for BoNT/A as low as about 0.1 pM to 1.1 pM for BoNT/A in optimized cell based assays performed by direct measurement of fluorescence from the cells as described above. Within the context of this application, an EC₅₀ should be understood as a concentration correspond to the midpoint of a sigmoidal dose response curve. Similarly, within the context of this application a lowest detectable dose (LDD) should be understood as a concentration corresponding to a response along a dose response curve that is distinguishable from a negative control (e.g., a test condition containing no compound to which a response is being tested, such as a BoNT or a surfactant). Such distinction can, for example, be determined as a response that deviates from the negative control condition by at least 2 standard deviations, or that provides clear demonstration of the beginning of a trend in the data.

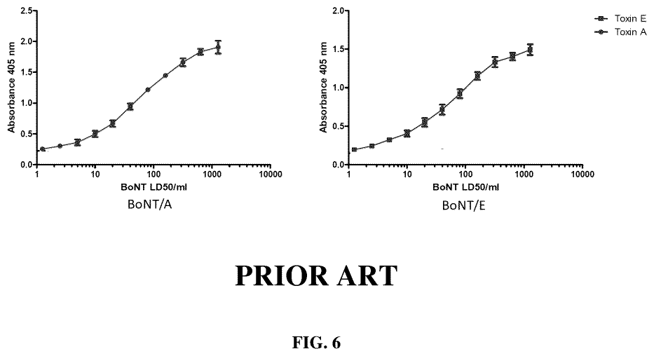

Notably, the human neuroblastoma cell line that acts as the parental cell line for the 5E9, 8F4, and 2C2 genetically modified cell lines has been shown to be equally responsive to BoNT/A and BoNT/E in regard to cleavage of SNAP25 as determined by immunoassay characterization of intoxicated cell contents. Cells of this human neuroblastoma cell line were treated with equivalent concentration ranges (as determined by a mouse lethality assay) of either BoNT/A or BoNT/E for 48 hours, lysed, and their contents characterized using an enzyme-linked immunoassay. Briefly, contents of lysed cells were added to test wells coated with antibodies directed to SNAP25 epitopes exposed by either BoNT/A or BoNT/E cleavage. Captured SNAP25 fragments were subsequently visualized using polyclonal antibodies directed to SNAP25 sites present in fragments produced by both BoNT/A and BoNT/E cleavage. Results are shown in FIG. 6. As shown, the human neuroblastoma cell line that was genetically modified to generate the 5E9, 8F4, and 2C2 cell lines does not discriminate between BoNT/A and BoNT/E.

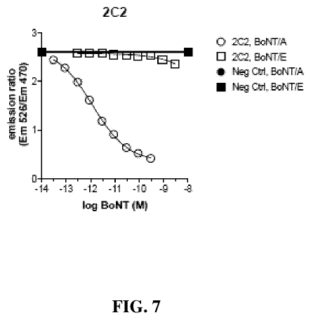

The reporting construct expressed by the genetically modified human neuronal cell line incorporates both BoNT/A and BoNT/E cleavage site and recognition sites derived from SNAP25 and can be cleaved by the proteolytic activity of either BoNT/A or BoNT/E. Cells from the 2C2 genetically modified human neuroblastoma cell line were exposed to various concentrations of BoNT/A and BoNT/E, and emission from both YFP and CFP measured using independent excitation measurements. As noted above, in such a cell-based assay YFP is released by BoNT/A or BoNT/E proteolysis and rapidly degraded in the cytosol, whereas CFP is retained on the cell membrane and is protected from proteolysis. Results of normalized emission (i.e., YFP emission divided by CFP emission) emission measurement from these cells are shown in FIG. 7. The genetically modified human neuronal cell line shows excellent sensitivity to BoNT/A, with the normalized emission measurements taken directly from the cells showing a lowest detectable dose (LDD, characterized as providing a distinguishable decrease from media without BoNT) using at little as 0.05 pM BoNT/A. Surprisingly, the same measurements made following exposure to BoNT/E show almost no response over the range tested (up to 50 nM). It should be appreciated that this high level of sensitivity to BoNT/A and high degree of discrimination between BoNT/A and BoNT/E was achieved by direct measurement of intact cells in culture and in the absence of serotype-specific neutralizing antibodies.

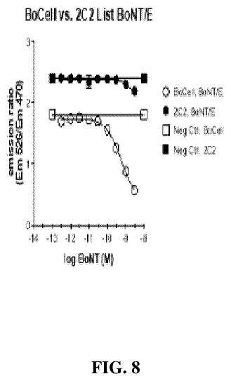

As noted above, BoCell® cells are a murine cell line expressing the same reporting construct that is expressed in the 2C2 cell line. FIG. 8 shows comparative BoNT/E dose response curves for BoCell® and 2C2 cell lines in cell based assays performed under the same testing conditions (time, temperature, etc.). As shown, the cells of the BoCell® cell line show a high degree of sensitivity to BoNT/E when compared to 2C2 cells.

Previous studies of the sensitivity of BoCell® to BoNT/A and BoNT/E in cell based assays performed at 37° C. show that the LDDs for BoNT/A and BoNT/E are similar, having values within 30% of each other. Since different cell lines transformed to express the same reporting construct show different degrees of serotype discrimination this unexpected finding cannot be attributed to the reporting construct itself. It is apparent that the response of a neuronal cell line to different botulinum neurotoxin serotypes having the same substrate specificity can be altered in the process of genetic modification to introduce a BoNT sensitive reporting construct.

Discrimination between different botulinum serotypes directed to the same substrate protein within such genetically modified cells can be sufficient to render the genetically modified cells essentially nonresponsive to one or more selected serotypes. For example, in the studies shown in FIGS. 7 and 8 the 2C2 cell line was unresponsive to BoNT/E to the point where an EC₅₀ could not be calculated, despite the use of high nM concentrations of the neurotoxin. Within the context of this application, genetically modified cells that are responsive to a target BoNT are essentially nonresponsive to a non-target BoNT serotype when a 1,000-fold, 2,00-fold, 3,000-fold, 5,000-fold, 8,000-fold, 10,000-fold or greater excess of the non-target BoNT is required to generate a response (e.g., an LDD) from the cell equivalent to that produced by the target BoNT. For example, as shown in FIG. 7, when 2C2 cells are exposed to BoNT/A or BoNT/E and approximately 0.5 nM of BoNT/E is required to produce a response that corresponds that provided by 0.05 pM of BoNT/A (i.e., an approximately 10,000-fold difference) under the same testing conditions. It should be appreciated that this degree of discrimination is observed in the absence of antibodies or antibody fragments specific for a BoNT serotype for which desensitization is desired. Similarly, this degree of discrimination is observed in the absence of competing BoNT heavy chain or analogs corresponding to a BoNT serotype for which desensitization is desired.

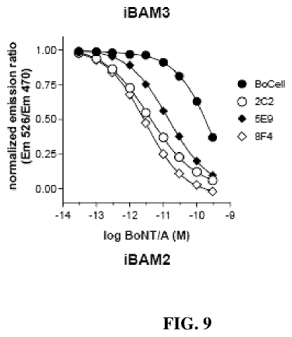

Further studies provided comparison in sensitivity of BoCell® transformed cells and 2C2 transformed cells expressing the same reporting construct to BoNT/E, in iBAM2+18 mM NaCl culture media. Comparative results of BoCell® and the genetically modified 2C2 cell line when exposed to BoNT/E are shown in FIG. 9. As shown, BoCell® retains sensitivity to BoNT/E whereas the genetically modified 2C2 cell line tested under the same conditions does not. Accordingly, differences in BoNT/E sensitivity are not due to the characteristics of the reporting construct. Accordingly, differences in BoNT/E sensitivity are not due to the characteristics of the reporting construct.

Studies were also performed comparing the sensitivity of BoCell® and 2C2, 5E9, and BF4 transformed cell lines responses to BoNT/A. Results are shown in FIG. 9. As shown, human neuroblastoma-derived transformed cell lines 2C2, 5E9, and BF4 show similar responses to increasing BoNT/A concentration, and these show increased response to low concentrations of BoNT/A (i.e., improved sensitivity) relative to those of BoCell® transformed with the same reporting construct.

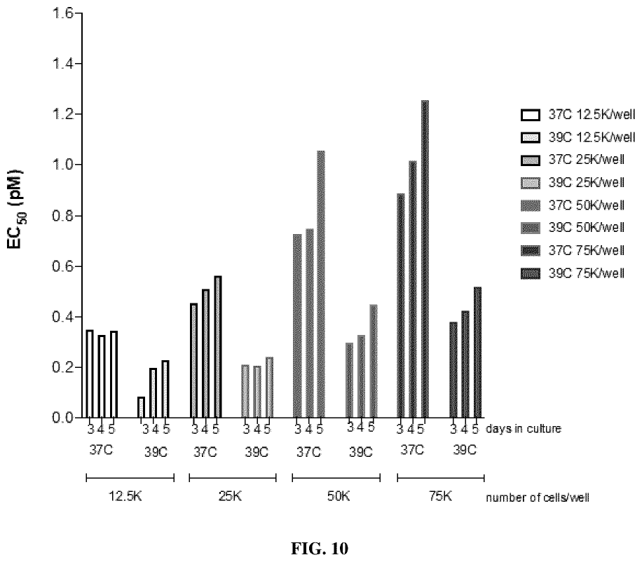

Cell-based assays based on genetically modified human neuroblastoma cell lines were shown to be impacted by the initial plating density of the cells, the number of days spent in culture, and the temperature at which the cells were cultured. A comparison of the EC₅₀ concentrations of BoNT/A (i.e., the concentration corresponding to that at which the normalized emission from the reporter region is at the midpoint of the dose/response curve) for a human neuroblastoma transformed cell line expressing a BoNT/A sensitive reporting construct are shown in FIG. 10.

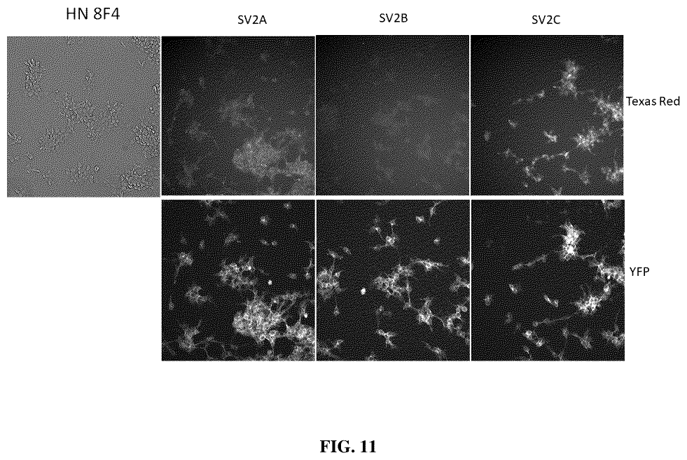

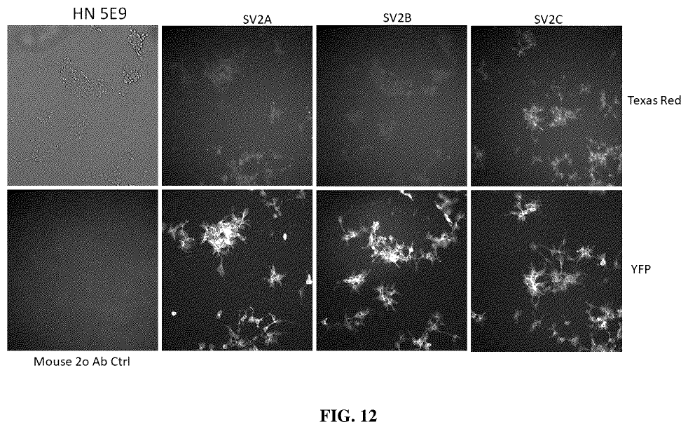

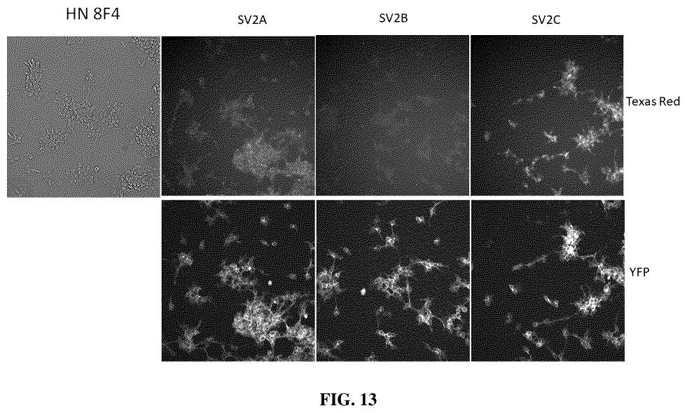

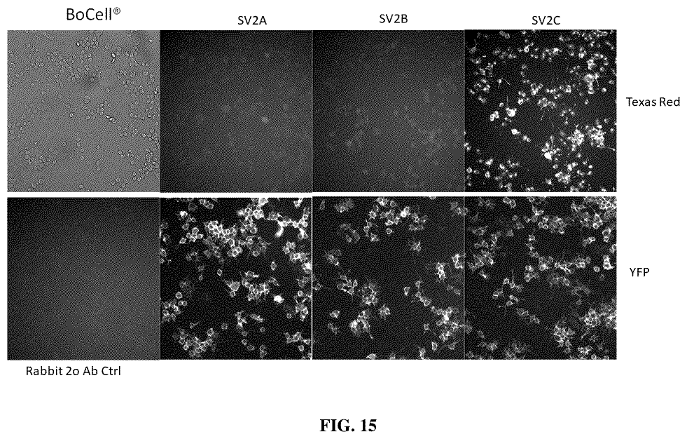

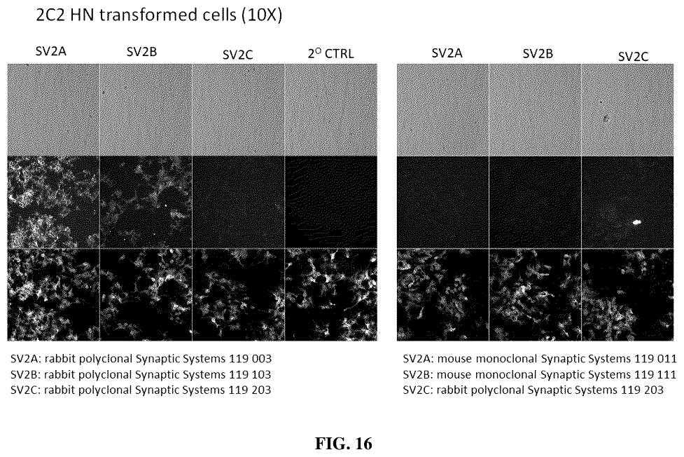

Generally, EC₅₀ values observed for BoNT/A assays performed using genetically modified human neuroblastoma cells can be lower than those observed for a murine BoCell® cell line engineered to express the same reporting construct. Inventors believe that at least some of the properties of genetically modified human neuroblastoma cells transformed to express BoNT-sensitive reporting constructs (as described above) are attributable to SV2 expression in these cells. FIGS. 10 to 13 show photomicrographs of human neuroblastoma cell lines (FIGS. 10 to 13) and a BoCell® cell line (FIG. 15) that express the same BoNT/A sensitive reporting constructs when stained with antibodies specific for SV2A (anti-SV2A mouse mAb 119 011), SV2B (anti-SV2B mouse mAb 119 111), and SV2C (anti-SV2C rabbit pAb 119 203). Cells can be visualized by YFP fluorescence produced by the reporting construct; specific antibodies are labeled with Texas Red. As shown, genetically modified human neuroblastoma cells show expression of SV2A, SV2B, and SV2C, with expression of SV2A and SV2C at roughly similar levels. In contrast, the murine BoCell® cell line expressing the same reporting construct shows SV2C expression almost exclusively. Additional studies performed using polyclonal antibodies directed to SV2 isoforms (SV2a, SV2b, SV2c) indicate that genetically modified human neuroblastoma cells can also show high expression of SV2b, as shown in FIG. 16.

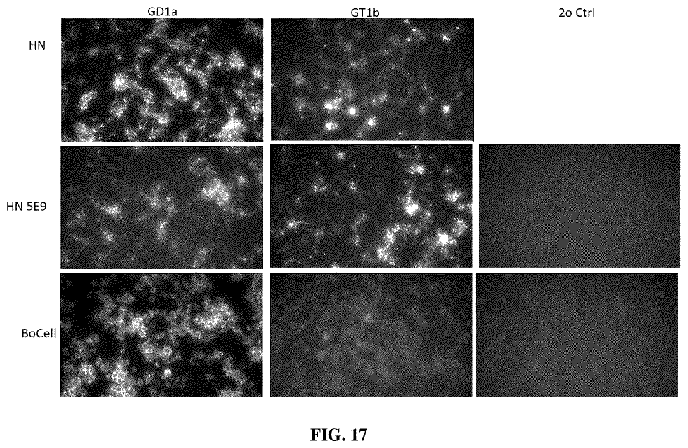

Similar studies were performed to characterize ganglioside content of human genetically modified neuroblastoma cell engineered to express a BoNT/A sensitive reporting construct and the BoCell® cell line expressing the same reporting construct. Gangliosides were visualized using anti-GD1a (mouse mAb, Millipore MAB5606Z or Anti-GT1b (mouse mAb, Millipore MAB5608). Results are shown in FIG. 17. As shown, genetically modified human neuroblastoma cells stain brightly for both GD1a and GT1b gangliosides, whereas BoCell® cells stain predominantly for GD1a.

Without wishing to be bound by theory, Inventors believe that selection of genetically modified human cell lines or genetically modified cell lines expressing a broader range of SV2 proteins and/or both GD1a and GDTb gangliosides can provide improved assay performance relative to murine cell lines in cell-based BONT assay, for example cell-based BONT assays in which the cells express a BoNT-sensitive reporting construct. For example, EC₅₀ in a culture media and culture condition optimized cell-based assay for a BoNT and performed using such genetically modified human cell lines (or genetically modified cell lines expressing a broader range of SV2 proteins and/or both GD1a and GDTb gangliosides) expressing a BoNT-sensitive reporting construct can have an EC₅₀ that is 90%, 80%, 70%, 60%, 50%, 40%, 30%, 10%, 5%, 3%, 1%, 0.5%, 0.3%, 0.1%, or less than an EC₅₀ measured from a culture media and culture condition optimized cell-based assay performed using murine cells transformed to express the same reporting construct.

As noted above, cell-based assays utilizing genetically modified cells of the inventive concept can provide BoNT assays with excellent sensitivity and serotype specificity. Such cell-based assays can be applied to any type of sample in which the presence of a BoNT is suspected or in which BoNT content needs to be characterized. Suitable sample types include foods, beverage, human or animal samples (e.g., tissue samples, blood, plasma, scrum, lymphatic fluid, tears, cerebrospinal fluid, saliva, urine, feces, etc.), and BoNT-containing pharmaceutical products. Such human or animal samples can be freshly obtained or preserved (e.g., by freezing or storage at reduced temperature). In some embodiments such human or animal samples can be treated to reduce degradation of BoNT content prior to testing, for example by the addition of a protease inhibitor.

Commercial BONT formulations (e.g., pharmaceuticals formulated for injection) frequently include excipients that are not generally considered compatible with cell based assays, such as surfactants, detergents, and peptides. Accordingly, where cell based assays are sensitive to such excipients they may need to be removed prior to testing of such pharmaceutical formulations for BONT content. These removal steps can include dialysis, diafiltration, size exclusion chromatography, etc., and may be only partially successful in the case of surfactants due to micelle formation. As such many pharmaceutical formulations may require extensive processing prior to characterization using cell based assays, which can in turn impact the accuracy and precision of such assays. Surprisingly, Inventors have found that genetically modified human neuroblastoma cells expressing a BoNT-sensitive reporting construct as described above are relatively tolerant of surfactants commonly used as excipients in BONT containing pharmaceuticals.

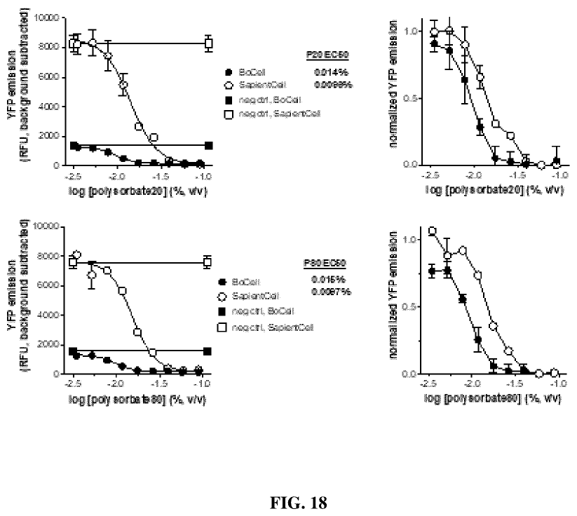

FIG. 18 shows the results of exposure of BoCell® cells and genetically modified human neuroblastoma cells expressing the same reporting construct to polysorbate 20 (P20, in the upper portion of FIG. 18) and polysorbate 80 (P80, in the lower portion of FIG. 18) at different concentrations. Cell viability was characterized by measuring fluorescence emissions of the fluorescent moieties of the reporting construct. The left side of FIG. 18 shows fluorescence emissions from the portion of reporting construct that is degraded upon exposure of the cells to BoNT/A. The right side of FIG. 18 shows fluorescence emissions from the cells normalized as described above. As shown, the genetically modified human neuroblastoma cells show an unexpected tolerance to both surfactants, which in combination with their high sensitivity for BoNT/A can permit simple dilution of BoNT/A containing pharmaceutical formulations prior to testing.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

What is claimed is:

1. A cell produced by genetic modification of a parental neuronal cell line and comprising a reporting construct, wherein the cell is greater than 100-fold more selective for intoxication by serotype A botulinum neurotoxin (BoNT/A) than by serotype E botulinum neurotoxin (BoNT/E), wherein the parental cell line is equally susceptible to both BoNT/A and BoNT/E, and wherein the reporting construct comprises a peptide that is a substrate for both BoNT/A and BoNT/E.

2. The cell of claim 1, wherein the parental neuronal cell line is a human neuronal or human neuroblastoma cell line.

3. The cell of claim 1, wherein the parental neuronal cell line is selected from the group consisting of LA-N-2, SH-SY5Y, N2a, SiMa, NS-20Y, NIE-115, NG108-C15, NSC-34, NSC-19, M4b, CNh, G4b, HT-22, and PC12.

4. The cell of one of claims 1 to 3, wherein the cell remains viable after exposure to polysorbate 20 or polysorbate 80 at a concentration of 0.017% w/v for 72 hours.

5. A method of selectively quantifying BoNT/A, comprising:
obtaining a first emission measurement from the cell of one of claims 1 to 4 at a first wavelength;
obtaining a second emission measurement from the cell at a second wavelength;
dividing the first measurement by the second measurement to obtain a first emission ratio;
contacting the cell with a solution comprising a botulinum neurotoxin;
after a period of time, obtaining a third measurement from the cell at the first wavelength and a fourth measurement from the cell at the second wavelength; and
dividing the third measurement by the fourth measurement to obtain a second emission ratio,
wherein reduction of the second emission ratio relative to the first emission ratio is indicative of the presence of a botulinum neurotoxin to which the cell is susceptible in the solution.

6. The method of claim 5, wherein the method has a limit of detection of 50 fM or less for BONT/A.

7. The method of claim 5 or 6, wherein the first emission ratio is not distinguishable from the second emission ratio when the botulinum neurotoxin is BoNT/E at a concentration of 0.5 nM.

8. The method of one of claims 5 to 7, wherein the solution further comprises one or more pharmaceutical excipients that are not removed prior to contacting with the cell.

9. The method of claim 8, wherein the one or more pharmaceutical excipients comprises a surfactant.

10. The method of claim 9, wherein the surfactant is polysorbate 20 or polysorbate 80.

11. The method of one of claims 5 to 10, wherein the period of time is 72 hours.

12. The method of one of claims 5 to 11, wherein the solution is a human or animal sample.

13. The method of claim 12, wherein the human or animal sample is selected from the group consisting of blood, serum, and plasma.

14. A method of improving a cell-based assay for a botulinum neurotoxin (BoNT), comprising:
transfecting a human neuroblastoma cell to generate a genetically modified cell expressing a reporting construct, wherein the genetically modified cell expresses a plurality of SV2 isoforms or both GD1a and GT1b gangliosides, and wherein a portion of the reporting construct is cleavable by a protease activity of the BoNT; and
contacting the genetically modified cell with the BoNT.

15. The method of claim 14, wherein the genetically modified cell expresses SV2A. SV2b, and SV2c.

16. The method of claim 14 or 15, wherein the genetically modified cell expresses a plurality of SV2 isoforms, GD1a, and GT1b.

17. The method of one of claims 14 to 16, wherein the reporting construct comprises:
a membrane binding portion;
a linking portion comprising a cleavage site, wherein the cleavage site is a substrate for the BoNT protease activity; and
a reporting portion comprising a labile reporter, wherein the reporting portion is coupled to the membrane binding portion via the linking portion, such that cleavage of the cleavage sites results in release of the labile reporter into the genetically modified cell's cytoplasm, and wherein the labile reporter is subject to proteolysis in the genetically modified cell's cytoplasm within a time course of the cell-based assay.

18. The method of claim 17, wherein the labile reporter is a first fluorophore.

19. The method of claim 18, wherein the first fluorophore is a fluorescent peptide.

20. The method of one of claims 14 to 19, wherein the reporting construct further comprises a reference reporter coupled to the membrane binding portion, wherein the reference reporter remains coupled to the membrane binding portion of cleavage of the cleavage site.

21. The method of claim 20, wherein the reference reporter is a second fluorophore.

22. The method of claim 20 or 21, wherein the reference reporter is not subject to degradation upon cleavage of the cleavage site.

23. The method of one of claims 14 to 22, wherein improving comprises reducing a first EC₅₀ of a culture medium and temperature optimized cell-based assay performed using the genetically modified cell by 50% relative to a second EC₅₀ of a culture medium and temperature optimized cell based assay performed using a murine cell transformed to express the reporting construct.


SEQUENCE LISTING (1 sequence)

SEQ ID NO: 1 - protein, 693 aa, synthetic construct
MVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYKSGLRSRAMAEDAMRNELEEMQRRADQLADESLESTRRMLQLVEESKDAGIRTLVMLDEQGEQLERIEEGMDQINKDMKEAEKNLTDLGKFCGLCVCPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSGSNSMVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK